(19) United States
(12) Patent Application Publication
(10) Pub. No.: US 2006/0217533 A1
(43) Pub. Date: Sep. 28, 2006

(54) Production of N-acyl compounds

(75) Inventors: Max Braun, Wedemark (DE); Johannes Eicher, Sehnde-Ilten (DE); Saskia Braukmueller, Sarstedt (DE)

Correspondence Address: CROWELL & MORING LLP;INTELLECTUAL PROPERTY GROUP, P.O. BOX 14300, WASHINGTON, DC, 20044-4300, US

(73) Assignee: Solvay Fluor GmbH, Hannover (DE)

(21) Appl. No.: 11/372,167

(22) Filed: Mar. 10, 2006

Related U.S. Application Data
(63) Continuation of application No. PCT/EP04/09248, filed on Aug. 18, 2004.

(30) Foreign Application Priority Data
Sep. 11, 2003 (DE) 103 42 261.7
Nov. 12, 2003 (DE) 103 53 116.5

(51) Int. Cl. C07K 1/02; C07C 231/02

(52) U.S. Cl. 530/333; 562/450; 564/133; 564/135

(57) ABSTRACT

A process for producing a compound containing an acylated nitrogen atom in which the adduct compound formed from an N-protonated cation and a chloride, bromide or iodide anion or the free nitrogen base is reacted with an acyl halide, preferably an acyl chloride, in the presence of an acid that drives off the released hydrogen chloride (HCl), hydrogen bromide (HBr) or hydrogen iodide (HI).

### CROSS REFERENCE TO RELATED APPLICATIONS

This application is a continuation of international patent application no. PCT/EP2004/009248, filed Aug. 18, 2004, designating the United States of America, and published in German as WO 2005/028420 on Mar. 31, 2005, the entire disclosure of which is incorporated herein by reference. Priority is claimed based on Federal Republic of Germany patent application nos. DE 103 42 261.7, filed Sep. 11, 2003, and DE 103 53 116.5, filed Nov. 12, 2003.

### BACKGROUND OF THE INVENTION

The invention relates to a process for producing N-acylated compounds.

The acylation of amines with carboxylic acid chlorides is a conventional reaction; see Methoden der organischen Chemie (Houben-Weyl), 4th edition (1958), volume XI/2, pages 10 to 11 and 30 to 34. In this reaction, hydrogen chloride is eliminated and binds to a second amine molecule, forming the amine hydrochloride. In this process, half the amine is consumed for binding the released hydrochloric acid. If the amine is to be completely converted into the amide, the released hydrogen chloride must be bound in some other manner, e.g., by adding alkali carbonate. As an alternative, pyridine may be used as the acid-binding agent. On page 34 of the cited textbook, it is noted that although acetyl chloride is a vigorously acting acetylating agent for amines, it is used less frequently than acetic anhydride because the latter is easier to handle and almost always brings the desired result.

### SUMMARY OF THE INVENTION

It is an object of the present invention to provide an improved process for producing N-acyl compounds.

Another object of the invention is to provide a simplified process for producing compounds by acylating nitrogen atoms.

These and other objects are achieved in accordance with the present invention by providing a process for producing a compound with an acylated nitrogen atom, comprising reacting an acyl halide with a nitrogen compound to form the corresponding N-acylated compound and a hydrogen halide; in which acyl represents an RC(O) group in which R represents a linear or branched alkyl with 1 to 6 carbon atoms, phenyl, linear or branched alkyl with 1 to 6 carbon atoms which is substituted with at least one halogen atom, or phenyl which is substituted with at least one halogen atom; the nitrogen compound is present in the form of its free base or is N-protonated and present in the form of a salt with a chloride, bromide or iodine anion; and the reaction is carried out in the presence of an acid of sufficient strength to expel the hydrogen halide from the reaction mixture.

The process according to the invention comprises the production of compounds with an acyl-substituted nitrogen atom, the parent compounds being the corresponding nitrogen compounds in the form of N-protonated adducts with a chloride, bromide or iodide anion and acyl halide, the acyl halide preferably being acyl chloride; the acylation is carried out in the presence of the carboxylic acid that corresponds to the acyl group used. The nitrogen atom must, in addition to the proton, have at least one additional hydrogen atom. Alternatively, it is possible to start from the free hydrogenous base, but in this case at least one hydrogen atom must be bound to the nitrogen atom.

As used herein, the term “acyl” represents the group RC(O), where R is linear or branched alkyl with 1 to 6 carbon atoms; phenyl; linear or branched alkyl with 1 to 6 carbon atoms which is substituted with one or more halogen atoms; or phenyl which is substituted with one or more halogen atoms. Preferably, acyl represents halogenated acyl, particularly trifluoroacetyl, difluoroacetyl, chlorodifluoroacetyl, C₂F₅C(O), C₂HF₄C(O) or C₂CIF₄C(O). Trifluoroacetylation is particularly preferred, the corresponding acid being trifluoroacetic acid.

Preferred acids in the presence of which the reaction is carried out thus include trifluoroacetic acid, difluoroacetic acid, chlorodifluoroacetic acid, pentafluoropropionic acid, tetrafluoropropionic acid, and chlorotetrafluoropropionic acid, with the acid preferably being selected to match the acyl group being added to the nitrogen-containing compound.

In principle, any nitrogen compounds may be acylated, such as amines or their hydrogen halide adducts or carboxylic acid amides. Preferred nitrogen compounds that are acylated using the process according to the invention include amino acids or their derivatives, e.g., the esters or peptides. The process according to the invention is particularly preferably used to produce N-acylated amino acid esters or peptides, especially compounds having the trifluoroacetyl group. Here, too, the hydrogen halide adducts, preferably the hydrochlorides, or the compounds with free N-atom may be used.

The process according to the invention is suitable for producing acyl amides, which may be used as such or in chemical synthesis. Trifluoroacetamide, which is a commercially available product, may be used in chemical synthesis, for example.

The introduction of an acyl group or halogenated acyl group is interesting also from the perspective of its property as a protective group for the nitrogen atom, particularly in chemical synthesis. The trifluoroacetyl group, in particular, is a known protective group; see, for example, U.S. Pat. No. 5,541,206, column 6.

The process according to the invention is particularly preferred for the acylation, particularly the trifluoroacetylation, of amino acids and their derivatives, especially the esters as well as peptides.

The process is particularly suitable for producing N-trifluoroacetyl-L-phenylalanine alkyl ester, N-trifluoroacetyl-D-phenylalanine alkyl ester and N-acyl-lysine alkyl esters (ω-amino group), particularly the methyl and ethyl esters. It is also possible to acylate esters, particularly alkyl esters, especially methyl and ethyl esters of glycine, alanine, valine, leucine, isoleucine, phenylalanine, tyrosine, proline, hydroproline, serine, threonine, cysteine, cystine, methionine, tryptophan, aspartic acid, glutamic acid, arginine and histidine.

The phenylalanine compounds are useful intermediates, for example in the production of the compounds mentioned in U.S. Pat. Nos. 5,541,206 and 4,816,484.

The acylated compounds obtained according to the invention may be isolated using conventional processes. A highly suitable process, particularly for the solidification of the compounds, is described in published US patent application no. 2005/0267290 (=WO 03/080563). A reaction mixture, containing impurities and an organic compound having a melting point greater than 30° C., is heated to evaporate the impurities. The remaining organic compound is then deposited onto a cooled running belt. This type of method may also be used for the compounds obtained by the present process.

The invention has the advantage that no ammonium halide is generated and needs to be disposed of.

The invention will be explained in further detail hereinafter with reference to the following illustrative working examples.

### EXAMPLES

Abbreviations:

MeOH=methanol
MeOH*HCl=solution of methanol and HCl
TFA=trifluoroacetic acid
TFAC=trifluoroacetyl chloride

### Example 1

### Production of L-phenylalanine Methyl Ester Hydrochloride from Phenylalanine

C₆H₅CH₂CH(NH₂)CO₂H+MeOH*HCl→C₆H₅CH₂CH(NH₂)CO₂CH₃*HCl L-phenylalanine L-phenylalanine methyl ester*HCl

Charge:

| | |
|---|---|
| 61.6 mole (1973.66 g) | MeOH (32.04 g/mole) |
| 9.8 mole (352.8 g) | HCl (36.0 g/mole) |
| 3.05 mole (503.83 g) | L-phenylalanine (165.19 g/mole) |
| 1.0 mole (103.0 g) | diisopropyl ether (102.18 g/mole) |

Procedure:

A 4 liter multi-neck flask was charged with the phenylalanine in the solvent, mixed with a previously prepared MeOH*HCl solution and dissolved. To improve dissolution the charge was heated to 50° C. and refluxed (at approximately 55° C.) for about 5 minutes. A vacuum was then applied to distill off the excess MeOH and the HCl. Distillation was continued until the charge became turbid, more viscous and formed white flocs. After having been allowed to stand, the precipitated phenylalanine methyl ester×HCl was suction filtered. The purity analyzed by gas chromatography (GC) was 99.4% with a melting point of 160° C.

Repeating the experiment with D- or D/L phenylalanine as the educt produced similar results.

### Example 2

### Trifluoroacylation of L-phenylalanine Methyl Ester Hydrochloride

C₆H₅CH₂CH(NH₂)CO₂CH₃*HCl in TFA+CF₃COCl→C₆H₅CH₂CH(NH—COCF₃)CO₂CH₃
L-phenylalanine methyl ester*HCl N-trifluoroacetyl-L-phenylalanine methyl ester

Charge:

| | |
|---|---|
| 2.0 mole (435.2 g) | L-phenylalanine methyl ester hydrochloride |
| | (215.68 g/mole) from Example 1 |
| 7.8 mole (889.36 g) | trifluoroacetic acid (114.02 g/mole) |
| 2.6 mole (350.5 g) | trifluoroacetyl chloride (132.47 g/mole) |

Procedure:

A 4 liter multi-neck flask was charged with the L-phenylalanine methyl ester (present as the HCl adduct) and dissolved in TFA. During the dissolution process, the charge was slowly heated to 90° C. This caused HCl to be released, detectable by the vigorous gas evolution at the bubble counter. After the ester had completely dissolved and the temperature was reached, TFAC was introduced, while a certain amount of HCl was still being released.

Introduction of TFAC was continued until all the ester had been converted into N-trifluoroacetyl-L-phenylalanine methyl ester. The solvent TFA was removed in vacuo. After cooling N-trifluoroacetyl-L-phenylalanine methyl ester having a melting point of 52° C. remained in a yield of 95% of the theoretical value.

In a variant of the experiment, the molten mass was directed onto a cooled belt. This resulted in an easy-to-handle product in the form of readily pourable pellets.

### Example 3

### Trifluoroacetylation of D-phenylalanine Methyl Ester*HCl and D/L-Phenylalanine Methyl Ester*HCl from Example 1

Example 2 was repeated using D-phenylalanine methyl ester*HCl and D/L-phenylalanine methyl ester*HCl from Example 1. The results corresponded to those of Example 2.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A process for producing a compound with an acylated nitrogen atom, said process comprising reacting an acyl halide with a nitrogen compound to form the corresponding N-acylated compound and a hydrogen halide; wherein:
acyl represents an RC(O) group in which R represents a linear or branched alkyl with 1 to 6 carbon atoms, phenyl, linear or branched alkyl with 1 to 6 carbon atoms which is substituted with at least one halogen atom, or phenyl which is substituted at least one halogen atom;
said nitrogen compound is present in the form of its free base or is N-protonated and present in the form of a salt with a chloride, bromide or iodine anion; and
the reaction is carried out in the presence of an acid of sufficient strength to expel the hydrogen halide from the reaction mixture.

2. A process according to claim 1, wherein the acyl group is a halogenated acyl group selected from the group consisting of trifluoroacetyl, difluoroacetyl, chlorodifluoroacetyl, C₂F₅C(O), C₂HF₄C(O) and C₂CIF₄C(O).

3. A process according to claim 2, wherein the reaction is carried out in the presence of a halogenated carboxylic acid corresponding to the halogenated acyl group.

4. A process according to claim 3, wherein the acyl group is a trifluoroacetyl group, and the reaction is carried out in the presence of trifluoroacetic acid.

5. A process according to claim 1, wherein the nitrogen compound is an amino acid, an ester of an amino acid, or a peptide.

6. A process according to claim 5, wherein the nitrogen compound is an L-phenylalanine alkyl ester, D-phenylalanine alkyl ester or lysine alkyl ester, and the acyl group is an N-trifluoroacetyl group.

7. A process according to claim 6, wherein the alkyl ester is a methyl ester or an ethyl ester.
